(19) United States
(12) Patent Application Publication
(10) Pub. No.: US 2002/0049992 A1
(43) Pub. Date: Apr. 25, 2002

(54) Modified promoters

(76) Inventor: Carol Hamilton, Apex, NC (US)

Correspondence Address: PARADIGM GENETICS, INC, 104 ALEXANDER DRIVE, BUILDING 2, P O BOX 14528, RTP, NC, 277094528

(21) Appl. No.: 09/887,384

(22) Filed: Jun. 22, 2001

Related U.S. Application Data
(63) Continuation-in-part of application No. 09/453,366, filed on Dec. 1, 1999.

(51) Int. Cl. A01H 5/00; C07H 21/04; C12N 5/04; C12N 15/74

(52) U.S. Cl. 800/278; 435/410; 536/23.4; 435/320.1

(57) ABSTRACT

Plant, seeds and DNA constructs containing DNA that is:

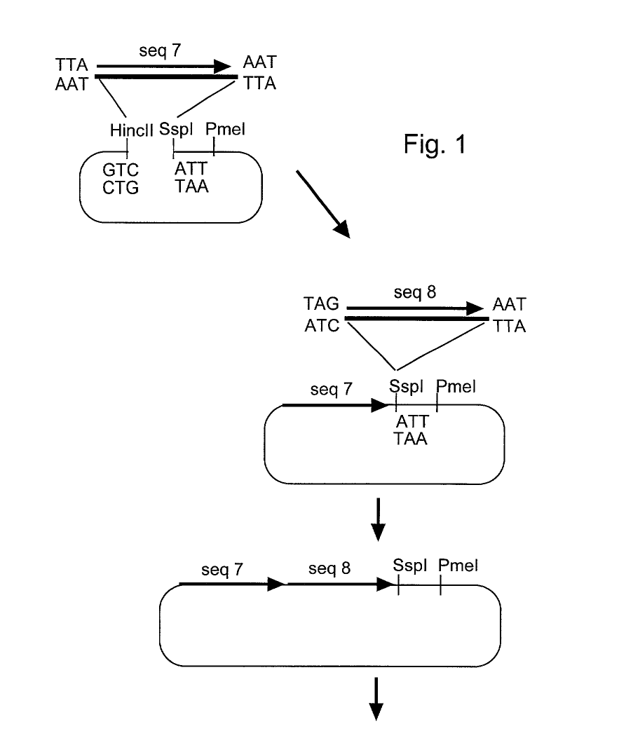

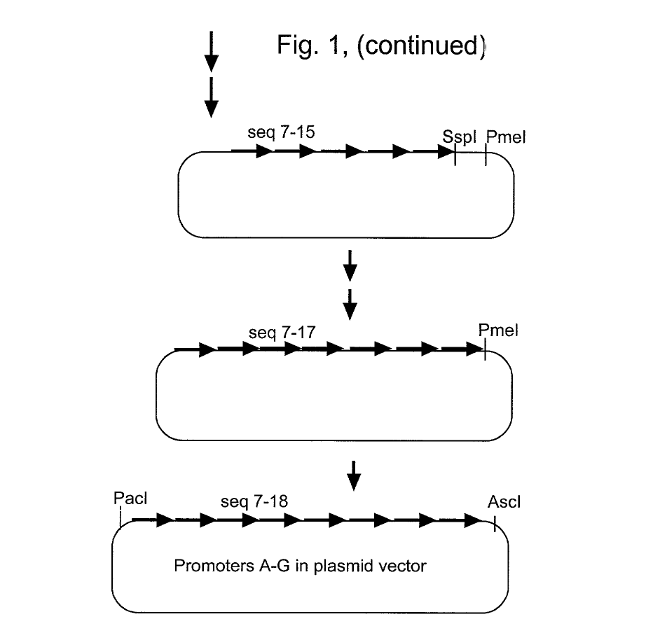

a) SEQ ID NO: 1, SEQ ID NO:2, SEQ ID NO:3, SEQ ID NO: 4, SEQ ID NO: 5, SEQ ID NO: 6;

b) the complementary sequence thereof, or

c) the double stranded sequence of a) and b).as well as a method for their preparation are disclosed herein.

### CROSS-REFERENCE TO RELATED INVENTION

This application is a continuation-in-part of copending U.S. Pat. application Ser. No. 09/453,366, filed on Dec. 1, 1999, the contents of which is incorporated by reference.

### FIELD OF THE INVENTION

The present invention relates to highly effective modified promoters for plants, such as soybeans and rice, and animals.

### BACKGROUND OF THE INVENTION

Recent advances in genetic engineering have provided plant breeders and geneticists with the tools to insert or transform genes, which are selected portions of deoxyribonucleic acid (also known as DNA), into a plant in order to produce new kinds of plants known as transgenic plants. Such transgenic plants or crops can have unique characteristics or traits, including resistance to plant diseases, resistance to herbicides, resistance to insects, enhanced stability or shelf-life of the ultimate consumer product obtained from the plant and/or improvements in the nutritional value in the edible portions of the plant. Genes are made up of DNA, a complex molecule inside each plant cell that provides the instructions for all aspects of the plant's growth. A promoter is a region on a gene where transcription factors can bind to enable the gene to “express” itself through the production of another, but smaller molecule known as messenger RNA. Messenger RNA enables the gene to “deliver” its message or instructions to other parts of the plant cell in many cases by being translated into a protein. Various plant promoters have been identified and isolated from different plants, as described in various patents, such as U.S. Patents 5,536,653; 5,589,583; 5,608,150; and 5,898,096. Although effective, such promoters have not been modified or optimized to provide enhanced or improved characteristics or traits. It would be desirable to provide plant promoters that have been modified to advantageously provide improved characteristics or traits in plants.

### SUMMARY OF THE INVENTION

The present invention relates to a modified promoter that when placed upstream of a gene of interest, will cause that gene to be expressed at a high level in plant vegetative tissues. The promoter should be active during most of the plant's developmental stages from the seedling stage to maturity.

In a first embodiment, the present invention is directed to a DNA molecule that is SEQ ID NO: 1, SEQ ID NO:2, SEQ ID NO:3, SEQ ID NO: 4, SEQ ID NO: 5 and SEQ ID NO: 6. In its double stranded form, SEQ ID NOS: 1-6 are useful as modified promoters in plants, such as dicots and monocots. For purposes of identifying the most concise promoter, SEQ ID NOS: 1 and 3 are preferred. For purposes of identifying the optimal modified promoter, SEQ ID NOS. 3 and 6 are preferred.

In a second embodiment, the present invention is directed toward a DNA construct comprising a DNA molecule that contains SEQ ID NO: 1, SEQ ID NO:2, SEQ ID NO:3, SEQ ID NO: 4, SEQ ID NO: 5 and SEQ ID NO: 6. Preferably the DNA construct is a plasmid.

In a third embodiment, the present invention is directed toward a eukaryotic cell comprising a DNA molecule that contains SEQ ID NO: 1, SEQ ID NO:2, SEQ ID NO:3, SEQ ID NO: 4, SEQ ID NO: 5 or SEQ ID NO: 6. Preferably, the eukaryotic cell is a plant cell. Also preferred is that the eukaryotic plant cell is a dicot plant cell. However, the eukaryotic plant cell may also be a monocot plant cell.

In a fourth embodiment, the present invention is directed toward a plant having eukaryotic cells comprising a DNA molecule that contains SEQ ID NO: 1, SEQ ID NO:2, SEQ ID NO:3, SEQ ID NO: 4, SEQ ID NO: 5 or SEQ ID NO: 6. Preferably, the plant is a dicot plant, although the plant may also be a monocot plant. Also preferred is that the dicot plant is Arabidopsis thaliana.

In a fifth embodiment, the present invention is directed toward seed capable of producing a plant having cells comprising a DNA molecule that contains SEQ ID NO: 1, SEQ ID NO:2, SEQ ID NO:3, SEQ ID NO: 4, SEQ ID NO: 5 or SEQ ID NO: 6. Preferably, the seed is capable of producing a plant that is a dicot plant, although the seed may also be able to produce a plant that is a monocot plant.

In a sixth embodiment, the present invention is directed toward a method of controlling and/or increasing the transcription of a heterologous or homologous gene in a plant or plant tissue comprising transforming the plant or plant tissue with a DNA construct comprising a heterologous gene and a DNA molecule that is SEQ ID NO: 1, SEQ ID NO:2, SEQ ID NO:3, SEQ ID NO: 4, SEQ ID NO: 5 or SEQ ID NO: 6.

### DETAILED DESCRIPTION OF THE INVENTION

The present invention will now be described more fully hereinafter with reference to the accompanying figures or drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs.

“Promoter” refers to the nucleotide sequences at the 5′ end of a structural gene which direct the initiation of transcription. Generally, promoter sequences are necessary to drive the expression of a downstream gene. The promoter binds RNA polymerase and accessory proteins, forming a complex that initiates transcription of the downstream nucleotide sequence. Some of the promoters of the present invention can also code for amino acids that will be fused to the protein gene product to yield fusion proteins. The additional amino acids are a conserved region at the beginning or N-terminal region of the actin 2 and actin 8 genes of Arabidopsis thaliana. The fusion proteins that are produced may be more stable in the plant cell than the non-fused version or counterpart protein. In the construction of heterologous promoter/structural gene combinations, the structural gene is placed under the regulatory control of a promoter such that the expression of the gene is controlled by promoter sequences. The promoter is positioned preferentially upstream of the structural gene, i.e., the amino acid coding region, and at a distance that approximates the distance between the promoter and the protein encoding region in its natural setting. As is known in the art, some variation in this distance can be tolerated without loss of promoter function.

The term “nucleic acid sequence” as used herein refers to a nucleotide, oligonucleotide, or polynucleotide, and fragments thereof, and to DNA or RNA of genomic or synthetic origin which may be single—or double—stranded, and which may represent a sense or antisense strand.

“Chemically synthesized,” as related to a sequence of DNA, means that the component nucleotides are assembled in vitro. Manual chemical synthesis of DNA may be accomplished using well established as known in the art.

“Gene” refers to a unit composed of a promoter region, a structural gene region and a transcription termination region.

“Expression” refers to the transcription and in the case of a protein gene product, translation, of a heterologous or homologous gene to yield the gene product encoded by the structural portion of the gene.

“Gene product” refers to a specific protein or RNA product derived from the structural portion of the gene.

“Heterologous” is used to indicate that a nucleic acid sequence (e.g., a gene) or a protein has a different natural origin or source with respect to its current host. Heterologous is also used to indicate that one or more of the domains present in a protein differ in their natural origin with respect to other domains present. In cases where a portion of a heterologous gene originates from a different organism the heterologous gene is also known as a chimera.

“Homologous” is used to indicate that a nucleic acid sequence (e.g. a gene) or a protein has a similar or the same natural origin or source with respect to its current host.

“Structural gene” is that portion of a gene comprising a DNA segment encoding the gene product, RNA or protein, and excluding the 5″sequence which drives the initiation of transcription. The structural gene may be one which is normally found in the cell or one which is not normally found in the cell wherein it is introduced, in which case it is termed a heterologous gene. A heterologous or homologous gene may be derived in whole or in part from any source known to the art, including a bacterial genome or episome, eukaryotic, nuclear or episomal DNA, organellar DNA e.g., mitochondrial or chloroplast DNA, cDNA, viral DNA or chemically synthesized DNA. A structural gene may contain one or more modifications in the coding region which could affect the chemical structure and/or the biological activity of gene product. Such modifications include, but are not limited to, mutations, insertions, deletions and substitutions of one or more nucleotides. The structural gene may constitute an uninterrupted coding sequence or it may include one or more introns, flanked by appropriate splice junctions. The structural gene may be a composite of segments derived from a plurality of sources, either naturally occurring or synthetic, or both.

“Gene product” refers to a specific protein or RNA product derived from the coding sequence.

“Transcription” is the process by which a downstream nucleotide sequence is “read” to produce a messenger RNA (mRNA). When the gene product is a specific protein, the mRNA is the molecule that is “read” by the translational machinery to produce that protein.

Variable regions at the beginning, i.e., 5′ end, and the end, i.e., 3′ end of the gene may or may not code for amino acids. Regions such as these are referred to as 5′ untranslated region (5′ UTR) and 3′ untranslated region (3′ UTR) respectively. A portion of the 5′ UTR serves as the binding region for the translational machinery (e.g., ribosomes and accessory proteins) required to synthesize a protein gene product encoded by an mRNA.

The promoter of the present invention set forth herein can be efficiently expressed in higher eukaryotes (e.g., plants), and more specifically will be more efficiently expressed in dicotyledenous plants, which include but are by no means limited to species of legumes (from the family Fabaceae), including soybean, peanut, and alfalfa; species of the Solanaceae family such as tomato, eggplant and potato; species of the family Brassicaceae such as cabbage, turnips and rapeseed; species of the family Rosaceae such as apples, pears and berries; and members of the families Cucurbitaceae (cucumbers), Chenopodiaceae (beets) and Umbelliferae (carrots).

The present invention provides an advantageously modified DNA promoter for the enhanced expression of desired heterologous or homologous protein genes in transgenic plants. To this end, one embodiment of the present invention is a DNA construct comprising a DNA sequence encoding the modified promoter. Such DNA constructs accordingly provide for the preparation of stably transformed cells expressing heterologous protein, which transformed cells are also an aspect of the invention. Still further, the modified promoters of the present invention provide for the subsequent regeneration of fertile, transgenic plants and progeny containing desired modified promoters. These aspects of the invention are further described herein below.

“DNA constructs” (also referred to herein as DNA vectors) of the present invention comprise the nucleotide sequence of the modified promoters, which nucleotide sequence is preferably the sequence provided herein as SEQ ID NOS:1-6. The preparation of DNA constructs is known in the art. See, e.g., Sambrook et al., Molecular Cloning: A Laboratory Manual (1989).

DNA constructs of the present invention contain the modified promoter for the expression of heterologous or homologous genes in plants. As used herein, the term “operatively linked” means that a promoter is connected to a coding region in such a way that the transcription of that coding region is controlled and regulated by that promoter.

The DNA sequences that comprise the DNA constructs of the present invention are preferably carried on suitable vectors, which are known in the art. Preferred vectors are plasmids that may be propagated in a plant cell. Particularly preferred vectors for transformation are those useful for transformation of plant cells or of Agrobacteria, as described further below. For Agrobacterium-mediated transformation, the preferred vector is a Ti-plasmid derived vector. For methods other than Agrobacterium-mediated transformation vectors which can be utilized as starting materials are known in the art.

Suitable vectors for transforming plant tissue and protoplasts have been described by deFramond, A. et al., Bio/Technology 1, 263 (1983); An, G. et al, EMBO J 4, 277 (1985);

and Rothstein, S. J. et al., Gene 53, 153 (1987). In addition to these, many other vectors have been described in the art which are suitable for use as starting materials in the present invention.

The DNA encoding the modified promoter of the present invention, and the DNA constructs comprising them, have applicability to any structural gene that is desired to be introduced into a plant to provide any desired characteristic in the plant, such as herbicide tolerance, virus tolerance, insect tolerance, disease tolerance, drought tolerance, or enhanced or improved phenotypic characteristics such as improved nutritional or processing characteristics.

Transgenes (heterologous or homologous genes transformed into a plant cell) will often be genes that direct the expression of a particular protein or polypeptide product, but they may also be non-expressible DNA segments, e.g., transposons that do not direct their own transposition. As used herein, an “expressible gene” is any gene that is capable of being transcribed into RNA (e.g., mRNA, antisense RNA, etc.) or translated into a protein, expressed as a trait of interest, or the like, etc., and is not limited to selectable, screenable or non-selectable marker genes. The invention also contemplates that, where both an expressible gene that is not necessarily a marker gene is employed in combination with a marker gene, one may employ the separate genes on either the same or different DNA segments for transformation. In the latter case, the different vectors are delivered concurrently to recipient cells to maximize cotransformation.

Any heterologous or homologous gene or nucleic acid that is desired to be expressed in a plant is suitable for the practice of the present invention. Heterologous and homologous genes to be transformed and expressed in the plants of the present invention include but are not limited to genes that encode resistance to diseases and insects, genes conferring nutritional value, genes conferring antifungal, antibacterial or antiviral activity, and the like.

Alternatively, therapeutic (e.g., for veterinary or medical uses) or immunogenic (e.g., for vaccination) peptides and proteins can be expressed in plants transformed with the modified promoters of the present invention. Alternately, plants may be transformed with one or more genes to reproduce enzymatic pathways for chemical synthesis or other industrial processes.

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the GUS gene). Of course, many examples of suitable marker genes are known to the art and can be employed in the practice of the invention. The selectable marker gene may be the only heterologous gene expressed by a transformed cell, or may be expressed in addition to another heterologous gene transformed into and expressed in the transformed cell. Selectable marker genes are utilized for the identification and selection of transformed cells or tissues. Selectable marker genes include genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS) respectively. Resistance to glufosinate ammonium, boromoxynil, and 2,4- dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes include, but are not limited to genes encoding resistance to: chloramphenicol (Herrera-Estrella et al., EMBO J 2, 987 (1983)); methotrexate (Herrera- Estrella et al., Nature 303, 209 (1983); Meijer et al., Plant Mol BioL 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); spectinomycin (Bretagne- Sagnard et al., Transgenic Res. 5, 131 (1 996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4- D (Streber et al., Bio/Technology 7, 811 (1989)); glufosinate-type herbicides, , such as phosphinothricin (PPT) or bialaphos (DeBlock et al., EMBO J 6, 2513 (1987)); spectinomycin (Bretagne-Sagnard and Chupeau, Transgenic Research 5, 131 (1996)). Other selectable markers that could be used in the vector constructs include, but are not limited to, the ALS gene for imidazolinone resistance, the EPSP synthase gene for glyphosate resistance, the Hm1 gene for resistance to the Hc-toxin, and other selective agents used routinely and known to one of ordinary skill in the art. See generally, Yarranton, Curr. Opin. Biotech. 3, 506 (1992); Chistopherson et al., Proc. Natl. Acad. Sci. USA 89, 6314 (1992); Yao et al., Cell 71, 63 (1992); Reznikoff, Mol Microbiol. 6, 2419 (1992); Barkley, et al., The Operon 177- 220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst et al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Baim et al., Proc. NatL Acad. Sci. USA 88, 5072 (1991); Wyborski et al., Nuc. Acids Res. 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J 2, 397 (1992); Gossen et al., Proc. Natl. Acad. Sci. USA 89, 5547 (1992); Oliva et al., Antimicrob. Agents Chemother. 36, 913 (1992); Hlavka et al., Handbook of Experimental Pharmacology 78, (1985); and Gill et al., Nature 334, 721 (1988). The disclosures described herein are incorporated by reference. The above list of selectable marker genes are not meant to be limiting. Any suitable selectable marker gene can be used in the present invention.

In view of the foregoing, it is apparent that one aspect of the present invention are transformed plant cells comprising the modified promoter of the present invention. “Transformation”, as defined herein, describes a process by which heterologous or homologous nucleic acid enters and changes a recipient cell. It may occur under natural or artificial conditions using various methods well known in the art. Transformation may rely on any known method for the insertion of foreign nucleic acid sequences into a eukaryotic host cell. Such “transformed” cells include stably transformed cells in which the inserted DNA is capable of replication either as an autonomously replicating plasmid or as part of the host chromosome. They also include cells which transiently express the inserted DNA or RNA for limited periods of time. In bacterial cell transformation, the promoter is carried on a plasmid vector as described herein. This plasmid is introduced into a bacterial host cell, such as E. coli or Agrobacterium tumefaciens or Agrobacterium rhizogenes or any other host that can support replication of the plasmid vector containing the desired promoter. Transformation can be achieved by using one of the methods known in the art, e.g., electroporation, heat shock, cold treatment, PEG-mediated transformation, biolistic transformation using a “gene gun.” Generally the plasmid carries a marker that can be used to select transformed cells. Selectable markers include but are not limited to antibiotic resistance markers such as kanamycin, ampicillin, tetracycline, gentamycin, chloramphenicol, hygromycin and to cytotoxic markers such as levan sucrase, cddB protein, and methotrexate. This generates a bacterial cell containing a plasmid that carries a gene that contains the desired promoter upstream of a structural gene of interest.

In a preferred embodiment of the invention, recipient cells for transformation are plant cells, preferably monocot plant cells, more preferably dicot plant cells, even more preferably Arabidopsis species plant cells, and most preferably Arabidopsis thaliana plant cells. “Plant cells” as used herein includes plant cells in plant tissue or plant tissue and plant cells and protoplasts in culture. Plant tissue includes differentiated and undifferentiated tissues of plants, including but not limited to, roots, shoots, leaves, pollen, seeds, tumor tissue and various forms of cells in culture, such as single cells, protoplasts, embryos and callus tissue. The plant tissue may be in plant, or in organ, tissue or cell culture. The recombinant DNA molecule carrying a structural gene under promoter control can be introduced into plant tissue by any means known to those skilled in the art. As novel means are developed for the stable insertion of foreign genes into plant cells and for manipulating the modified cells, skilled artisans will be able to select from such means to achieve a desired result. Means for introducing recombinant DNA into plant tissue include, but are not limited to, direct DNA uptake (Paszkowski, J. et al. (1984) EMBO J 3,2717), electroporation (Fromm, M., et al. Proc. NatL Acad. Sci. USA 82,5824 (1985), microinjection (Crossway, A. et al Mol. Gen. Genet. 202, 179 (1986)), or T-DNA mediated transfer from Agrobacterium tumefaciens to the plant tissue, which techniques are known in the art. There appears to be no fundamental limitation of T-DNA transformation to the natural host range of Agrobacterium.

Representative T-DNA vector systems are described in the following references: An, G. et al. EMBO J 4, 277 (1985); Herrera-Estrella, L. et al., Nature 303, 209 (1983); Herrera-Estrella, L. et al. EMBO J 2, 987 (1983); Herrera-Estrella, L. et al. in Plant Genetic Engineering, New York: Cambridge University Press, p. 63 (1985). In some methods, plasmid DNA is used to transfer the gene of interest to the plant. Markers that can be used to select for transformed plants include resistance to the following antibiotics: hygromycin, spectinomycin, phosphinotricin, kanamycin, methotrexate and the like. Methods that can be used to directly transfer the plasmid DNA to the plant cell include, PEG mediated transformation and biolistic transformation. Alternatively, a bacterial cell as described above can be to transfer the gene of interest to the plant. The most commonly used method that makes use of a bacterial cell is Agrobacterium-mediated plant transformation. Generally this method involves contact between or some type of plant tissue or a whole plant and a liquid suspension of Agrobacterium spp. that contains the plasmid with the gene of interest. This is followed by selection to identify plant tissues that have been transformed with the plasmid DNA or transformed seeds. Mature plants can be generated from transformed plant tissue via tissue culture or by germinating the seed of transformed plants. In either case the transformed plants are identified by their ability to grow on media that contains an antibiotic or other selective agent. Plants that are generated from tissue culture are grown to maturity and seed is collected to determine whether or not the gene of interest, the transgene, has been introduced into the germline of the plant. Once introduced into the plant tissue, the expression of the structural gene may be assayed by any means known to the art, and expression may be measured as mRNA transcribed or as protein synthesized, as provided herein.

Transgenic plants comprising the modified promoter of the present invention (as present, for example, in a DNA construct of the present invention, or a transformed cells of the present invention) are also an aspect of the present invention. Procedures for cultivating transformed cells to useful cultivars are known to those skilled in the art. Techniques are known for the in vitro culture of plant tissue, and in a number of cases, for regeneration into whole plants. A further aspect of the invention are plant tissue, plants or seeds containing the chimeric DNA sequences described above. Preferred are plant tissues, plants or seeds containing those chimeric DNA sequences which are mentioned as being preferred. The transformed cells, identified by selection or screening and cultured in an appropriate medium that supports regeneration as provided herein, will then be allowed to mature into plants. Plants are preferably matured either in a growth chamber or greenhouse. Plants are regenerated from about 6 weeks to 10 months after a transformant is identified, depending on the initial tissue. During regeneration, cells are grown on solid media in tissue culture vessels.

Illustrative embodiments of such vessels are petri dishes and Plant Con®s. After the regenerating plants have reached the stage of shoot and root development, they may be transferred to a greenhouse for further growth and testing. Progeny may be recovered from the transformed plants and tested for expression of the transgene by localized application of an appropriate substrate to plant parts such as leaves to assay for transgene activity or by observed complementation of a phenotype.

The regenerated plants are screened for transformation by standard methods illustrated below. Progeny of the regenerated plants are continuously screened and selected for the continued presence of the integrated DNA sequence in order to develop improved plant and seed lines. The DNA sequence can be moved into other genetic lines by a variety of techniques, including classical breeding, protoplast fusion or microinjection.

After effecting delivery of the promoter and heterologous or homologous DNA to recipient cells and plants by any of the methods discussed above it is generally useful to identify the cells exhibiting successful or enhanced expression of a heterologous gene for further culturing and plant regeneration. As mentioned above, in order to improve the ability to identify transformants, one may choose to employ a selectable or screenable marker gene as, or in addition to, the expressible gene of interest. In this case, one would then generally assay the potentially transformed cell population by exposing the cells to a selective agent or agents, and/or one would screen the cells for the desired marker gene.

“Screening” generally refers to identifying the cells exhibiting expression of a heterologous or homologous gene that has been transformed into the plant. Screening is carried out by assaying for the expression of a particular marker gene product, e.g., GUS, or by observation of a phenotype. To additionally confirm the presence of the heterologous or homologous nucleic acid or “transgene(s)” in regenerated plants, in the seeds of transformed or regenerated plants, or the in plants produced from those transgenic seeds, a variety of assays may be performed. Such assays include, for example, molecular biological assays, such as Southern and Northern blotting and PCR; biochemical assays, such as detecting the presence of a protein product, e.g., by immunological means (ELISAs and Western blots) or by enzymatic function; by plant part assays, such as leaf or root assays; and also, by analyzing the phenotype of the whole regenerated plant.

While Southern blotting and PCR may be used to detect the gene(s) in question, they do not necessarily provide information as to whether or not the gene is being expressed. Expression of a heterologous or homologous protein gene product may be evaluated by assaying for the protein products of the introduced genes or by evaluating the phenotypic changes brought about by their expression. Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis two-dimensional gel protein electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these techniques are among the most commonly employed, other procedures are known in the art and may be additionally used.

The term “restriction site” refers to a deoxyribonucleic sequence at which a specific restriction endonuclease cleaves the plasmid, vector or DNA molecule.

The modified promoter can be used to create a chimeric gene. The chimeric gene can then be tranformed into a plant by any practicable method. For example, in the transformed plant, the promoter can confer high level transcription of the downstream nucleotide sequence or of the contiguous structural coding in most plant cells, plant parts or plant tissues. Plant parts include attached or detached portions of a plant, including leaves, stems, roots, flowers, fruits or parts thereof.

The initial suspension of Agrobacterium is cultivated or grown until growth of Agrobacterium in the suspension is substantially completed, i.e. the density of bacteria in the suspension has reached near-maximum levels over time. Typically, the growth of Agrobacterium in the suspension is substantially completed after 16 to 24 hours, although longer or shorter times may be used. The optical density of the initial suspension can range from about 1.2 to about 2.4 or greater.

After the initial suspension of Agrobacterium is grown or formed, it is diluted with an aqueous medium to reduce the Agrobacterium and any other components in the growth medium (i.e. antibiotics) to a concentration that will allow the Agrobacterium to infect the plant without harming it. Typically, the initial suspension can be diluted by mixing from about 2 to about 10 volumes of aqueous medium per one volume of initial suspension, preferably from about 3 to about 5 volumes of aqueous medium. The optical density of the diluted suspension is less than 2, preferably from about 0.6 to about 1.5, more preferably from about 0.8 to about 1.0.

The aqueous medium used to dilute the initial suspension may optionally contain adjuvants or additives which can promote or enhance transformation by Agrobacterium, such as sugars and surfactants. Suitable sugars can include sucrose, fructose, glucose, galactose and the like. When sugars are used, the concentration of sugar in the aqueous medium can range from about 2 to about 10% by weight, preferably about 5% by weight.

The term “surfactant” or “surface-active agent” refers to any compound that can reduce surface tension when dissolved in water or water solutions or that can reduce interfacial tension between a liquid (water) and a solid (bacteria). Generally, the surfactant should not be harmful to the plant. Suitable surfactants that can be used in the aqueous medium can include Triton™ brand of surfactants, the Tween™ brand of surfactants and the Silwet™brand of surfactants. The Triton™brand of surfactants includes specialty surfactants that are alcohols and ethoxylates, alkoxylates, sulfates, sulfonates, sulfonosuccinates or phosphate esters. One preferred surfactant is Triton™X-100 (t-Octylphenoxypolyethoxyethanol) a widely used non-ionic surfactant. Another preferred surfactant is Silwet-L77® (polyalkyleneoxide modified heptamethyltrisiloxane). The concentration of surfactant in the aqueous medium can range from about 0.001 to about 0.5% 25 by weight, preferably from about 0.01 to about 0.05%.

To transform or treat the plant, the flower or bolt is contacted with the diluted suspension of Agrobacterium. For example, the flower can be dipped into diluted suspension containing the Agrobacterium for about 10 seconds to one minute or more. Alternatively, the diluted suspension of Agrobacterium can be sprayed or painted onto the flower portions of the plant.

After the plants have been treated with the diluted suspension of Agrobacterium, they are typically placed for one day into a dark room or chamber having a high relative humidity to encourage the Agrobacteriun to infect or transform the plant. The temperature is maintained from about 20 to about 30 degrees Celcius, preferably from about 22 to about 25 degrees Celcius (room temperature).

Optionally, and preferably, the treated plants are grown or cultivated under normal growth conditions to produce seed or to seed maturity.

Optionally, and preferably, seed that is collected or harvested may be grown into plants. Plants that have been transformed, i.e. that are transgenic due to the insertion of recombinant DNA or DNA of interest into their genome, may be selected by treating the plants with an antibiotic or herbicide and selecting those with antibiotic or herbicide resistance, an indicator of transfomation.

The present method can be used without a vacuum or with a vacuum as described in Bechtold et al., Methods Mol. Biol. 82:259-266 (1998).

A diagram showing the procedure for cloning of the modified promoter procedure into a suitable DNA construct is shown below. selected to have unique cloning sites in the following order: HincII, SspI and PmeI. The first restriction site (e.g. HincII) can alternatively be any restriction endonuclease that cleaves to generate blunt-ended fragments and is unique in the vector. The additional sites (e.g. SspI and PmeI) are chosen in be compatible with the polynucleotides. Cloning into the chosen unique sites in the vector should not interfere with normal plasmid function and propagation. Additionally, there should be no PacI or AscI (or analagous chosen rare-cutter) restriction sites present in the vector in order to allow easy subcloning of the promoter.

First, the promoter is divided into fragments. A fragment is defined as a double-stranded piece of DNA that is made by annealing two complementary polynucleotides. The polynucleotides are designed as complementary pairs of sequences that range in length from about 40 to about 100 polynucleotides. The polynucleotides are solubilized, complementary pairs are mixed in equal amounts, heated and then cooled slowly to anneal them together to make the double-stranded fragment. The fragments 1-12 are made up of polynucleotide sequences numbered 7-18 (shown) and their complementary polynucleotides 19-30 (not shown). That is, polynucleotide sequences 7 and 19 are annealled together to generate fragment 1. Sequences 8 and 20 are annealled together to generate fragment 2 and so on. The fragments thus made are joined or ligated together to create the promoter. One set of criteria for choosing the fragments that make up the promoter are as follows.

Fragment 1 (not shown) is made by annealing polynucleotide, sequence 7 and its complementary polynucleotide, sequence 19. Fragment 1 contains nucleotide sequences that create a recognition site for a rare cutting restriction endonuclease such as PacI located near the 5′ end of sequence 7. Polynucleotide sequence 7 is 74 nucleotides in length and ends with nucleotides AAT.

| | |
|---|---|
| TTAATTAATCGAGCAGGTCACAGTCATGAAGCCATCAAAGCAAAAGAACTAATC | [sequence 7] |
| CAAGGGCTGAGATGATTAAT | |

Fragment 2 (not shown) is made by annealing polynucleotide, sequence 8 and its complementary polynucleotide, sequence 20. Polynucleotide sequence 8 begins with the nucleotides immediately downstream or adjacent to the TAA of sequence 7 located at the 3′ end. Sequence 8 is 77 nucleotides in length and ends with nucleotides AAT.

| | |
|---|---|
| TAGTTTAAAAATTAGATAACACGAGGGAAAAGGCTGTCTGACAGCCAGGTCACG | [sequence 8] |
| TTATCTTTACCTGTGGTCGAAAT | |

Fragments 3 - 9 inclusive (not shown), are prepared in a similar fashion according to the procedure described above for fragment 2, except that sequences 9-15 and their corresponding complementary polynucleotides are used.

| | |
|---|---|
| GATTCGTGTCTGTCGATTTTAATTATTTTTTTGAAAGGCCGAAAATAAAGTTGTAA | [sequence 9] |
| GAGATAAACCCGCCTATATAAAT | |
| TCATATATTTTCCTCTCCGCTTTGAATTGTCTCGTTGTCCTCCTCACTTTCATCAGC | [sequence 10] |
| CGTTTTGAAT | |
| CTCCGGCGACTTGACAGAGAAGAACAAGGAAGAGAGAGAAAGTAAGAGATAAT | [sequence 11] |
| CCAGGTTCTCCGTTTTGAAT | |
| CTT CCT CAA TCT CAT CTT CTT CCG CTC TTT CTT TCC AAG GTA ATA GGA | [sequence 12] |
| ACT TTC TGG ATC TAC TTT ATT TGC TGG ATC TCG ATC TTG TTT TCT CAA T | |
| TTCCTTGAGATCTGGTTTTCGTTTAATTTGGATGGAATTTAGATCACTAAATCTTT | [sequence 13] |
| TGGTTTTACTAGAAT | |
| CGATCTAAGTTGACCGATCAGTTAGCTCGATTATAGCTACCAGAATTTGGCTTGA | [sequence 14] |
| CCTTGATGGAGAGATCCATGTTCATGTTACCTGGGAAAT | |
| GATTTGTATATGTGAATTGAAATCTGAACTGTTGAAGTTAGATTATTGACTGTAA | [sequence 15] |
| CTGTCAAT | |

Fragments 10 and 11 (not shown) are each made by annealing polynucleotide, sequences 16 and 17 with their respective complementary polynucleotide, sequences 28 and 29. Sequences 16 and 17 each begin with the nucleotides downstream or adjacent to the 3' end of the previous sequence. Sequences 16 and 17 each end with nucleotides GTTT at the 3′ end.

| | |
|---|---|
| GTTAGATTATTGACTGTAACTGTTTAAGTTAGATGAAGTTT | [sequence 16] |
| GTGTATAGATTCTTCGAAACTTTAGGATTTGTAGTGTCGTACGTTGAACAGAAAG | [sequence 17] |
| CTATTTCTGATTCAATCAGGGTTT | |

Fragment 12 (not shown) is made by annealing polynucleotide, sequence 18 and its complementary polynucleotide, sequence 30. Poynucleotide sequence 30 begins with the nucleotides immediately downstream or adjacent to the 3′ GTTT of sequence 17. Fragment 12 contains nucleotide sequences that create a recognition site for a rare cutting restriction endonuclease such as AscI located near the 3′ end of sequence 18. ATTTGACTGTATTGAACTCTTTTTGTGTGTTTGCAGCTCATAAACCGGCGCGCC [sequence 18]

Each pair of annealed polynucleotides is introduced sequentially into a standard cloning vector by DNA ligation. The first fragment is introduced between the PacI and SspI sites. The desired orientation of fragment 1 can be established by restriction enzyme analysis. The desired orientation places the 3′ AAT nucleotides of fragment 1 adjacent to the half of the SspI site that is on the vector DNA fragment, and recreates a SspI site.

The second fragment is inserted into the unique, recreated SspI site at the 3' end of fragment 1. The desired orientation of fragment 2 places the 3' AAT nucleotides of fragment 2 adjacent to the half of the SspI site that is on the vector DNA fragment, and recreates a SspI site. The third, fourth, fifth, sixth, seventh, eighth and ninth fragments are added sequentially exactly as described for fragment 2.

To add the tenth fragment the vector is cut with SspI and PmeI. The desired orientation of fragment 10 places the 3′ GTTT of fragment 10 adjacent to the half of the PmeI site that is on the vector DNA fragment and recreates a PmeI site.

The eleventh fragment is inserted into the unique, recreated PmeI site at the 3′ end of fragment 10. The desired orientation of fragment 11 places the 3′ GTTT nucleotides of fragment 11 adjacent to the half of the PmeI site that is on the vector DNA fragment and recreates a PmeI site. Fragment 12 is then introduced into the unique, recreated PmeI site. The entire promoter can then be removed from the vector by cutting with restriction enzymes PacI and AscI.

Promoters B, C, D, E, F and G can be prepared in accordance with a similar or analagous procedures such as those described herein.

Example B

This promoter has 1202 ′ nucleotides, as represented by SEQ ID NO. 2.

Example C

This promoter has 1285 nucleotides as represented by SEQ ID NO.3.

Example D

This promoter has 910 nucleotides, including 57 nucleotides that encode for 19 amino acids, as represented by SEQ ID NO.4.

Example E

This promoter has 1259 nucleotides, including 57 nucleotides that encode for 19 amino acids, as represented by SEQ ID NO. 5. The protein expressed from the gene driven by this promoter is a fusion protein. The fusion protein consists of the first 19 N-terminal amino acids of the actin 2 protein from Arabidopsis covalently linked to any protein that is encoded downstream. These 19 amino acids are conserved in actin 2 and actin 8 in Arabidopsis and are covalently linked to the N-terminal portion of the expressed protein of interest. This promoter construct potentially generates a more stable protein product. That is, the expressed protein of interest may be stablized by having its rate of degradation reduced compared to the expressed gene of interested alone, i.e., without the actin portion of the fusion protein.

Example F

This promoter has 1342 nucleotides, including is 57 nucleotides that encode for 19 amino acids, as represented by SEQ ID NO. 6. The gene product from this promoter is a fusion protein of 19 amino acids from actin that are covalently linked to the N-terminal portion of the expressed protein of interest. This promoter construct potentially generates a more stable protein product. That is, the expressed protein of interest may be stablized by having its rate of degradation reduced compared to the expressed gene of interested alone, i.e., without the actin portion of the fusion protein.

Preparation of Starting Materials

Synthetic oligonucleotides and polynuleotides of approximately 100 bases pairs or less, are commercially available. That is polynucleotides such as those used to prepare the promoters (described previously) are commercially available or can be prepared according to known procedures. Complementary polynucleotides are solubilized and equal molar amounts are heated and then cooled slowly to promote annealing.

What is claimed is:

1. DNA that is
a) SEQ ID NO: 1, SEQ ID NO:2, SEQ ID NO:3, SEQ ID NO: 4, SEQ ID NO: 5 or SEQ ID NO: 6;
b) the complementary sequence thereof; or
c) the double stranded sequence of a) and b).

2. DNA of claim 1 that is SEQ ID NO: 3 or SEQ ID NO: 6.

3. A DNA construct comprising DNA that is
a) SEQ ID NO: 1, SEQ ID NO:2, SEQ ID NO:3, SEQ ID NO: 4, SEQ ID NO: 5 or SEQ ID NO: 6;
b) the complementary sequence thereof; or
c) the double stranded sequence of a) and b).

4. The DNA construct of claim 3 that is SEQ ID NO: 3 or SEQ ID NO: 6.

5. The DNA construct of claim 3 that is the plasmid.

6. A eukaryotic cell comprising DNA that is
a) SEQ ID NO: 1, SEQ ID NO:2, SEQ ID NO:3, SEQ ID NO: 4, SEQ ID NO: 5 or SEQ ID NO: 6;
b) the complementary sequence thereof; or
c) the double stranded sequence of a) and b).

7. The eukaryotic cell of claim 6 comprising a DNA that is SEQ ID NO: 3 or SEQ ID NO: 6.

8. The eukaryotic cell of claim 6 wherein the cell is a plant cell.

9. The eukaryotic cell of claim 8 wherein the cell is a dicot plant cell.

10. The eukayrotic cell of claim 6 wherein the cell is a monocot plant cell.

11. A plant or plant part having a eukaryotic cell comprising DNA that is
a) SEQ ID NO: 1, SEQ ID NO:2, SEQ ID NO:3, SEQ ID NO: 4, SEQ ID NO: 5 or SEQ ID NO: 6;
b) the complementary sequence thereof; or
c) the double stranded sequence of a) and b).

12. The plant or plant part of claim 11 having a eukaryotic cell comprising a DNA that is SEQ ID NO: 3 or SEQ ID NO: 6.

13. The plant or plant part of claim 11 that is, or is from, a dicot plant.

14. The plant or plant part of claim 11 that is, or is from, a monocot plant.

15. The plant or plant part of claim 11 that is, or is from, Arabidopsis thaliana or Oryza sativa.

16. Seed that can produce a plant containing DNA that is:
a) SEQ ID NO: 1, SEQ ID NO:2, SEQ ID NO:3, SEQ ID NO: 4, SEQ ID NO: 5 or SEQ ID NO: 6;
b) the complementary sequence thereof; or
c) the double stranded sequence of a) and b).

17. Seed of claim 16 wherein the DNA is SEQ ID NO: 3 or SEQ ID NO: 6.

18. Seed from a plant of claim 11.

19. A method of regulating the transcription of a heterologous gene in a plant or plant tissue comprising transforming the plant or plant tissue with a DNA construct comprising a heterologous gene and DNA that is:
a) SEQ ID NO: 1, SEQ ID NO:2, SEQ ID NO:3, SEQ ID NO: 4, SEQ ID NO: 5 or SEQ ID NO: 6;
b) the complementary sequence thereof, or
c) the double stranded sequence of a) and b).


SEQUENCE LISTING (18 sequences)

SEQ ID NO: 1 - DNA, 853 bp, Artificial Sequence
  Description of Artificial Sequence  promoter
tcgagcaggtcacagtcatgaagccatcaaagcaaaagaactaatccaagggctgagatgattaattagtttaaaaattagataacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagagagagaaagtaagagataatccaggttctccgttttgaatcttcctcaatctcatcttcttccgctctttctttccaaggtaataggaactttctggatctactttatttgctggatctcgatcttgttttctcaatttccttgagatctggttttcgtttaatttggatggaatttagatcactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaacc

SEQ ID NO: 2 - DNA, 1202 bp, Artificial Sequence
  Description of Artificial SequenceActin based promoter
caactatttttatgtatgcaagagtttcatcttgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggaaatgaaaagtttaagataaccactaaaaaaacggagctgtcatgtaacacgcggatcgagcaggtcacagtcatgaagccatcaaagcaaaagaactaatccaagggctgagatgattaattagtttaaaaattagataacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagagagagaaagtaagagataatccaggttctccgttttgaatcttcctcaatctcatcttcttccgctctttctttccaaggtaataggaactttctggatctactttatttgctggatctcgatcttgttttctcaatttccttgagatctggttttcgtttaatttggatggaatttagatcactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaacc

SEQ ID NO: 3 - DNA, 1285 bp, Artificial Sequence
  Description of Artificial SequenceActin based promoter
ccatttttcttttgcatcaactatttttatgtatgcaagagtttcatcttgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaatttgtagaaatgatacaattctaatagaaaacgaattaaattacgttgaattgtatgaaatctaattgaacaagccaaccacgacgacgactaacgttgcctggaaatgaaaagtttaagataaccactaaaaaaacggagctgtcatgtaacacgcggatcgagcaggtcacagtcaatccatgaagccatcaaagcaaaagaactaatccaagggctgagatgattaattagtttaaaaattagataacacgagggaaaaggctgtctgacagccaggtcacgttatactctttacctgtggtcgaaatgattcgtgtctgtcgattttaattatttccaaaattgattttgaaaggccgaaaataaagttgtaagagataaacccgcctatataaattcatcaacactcgatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttcgtggatctactttatttgctggatctcgatcttgttttctcaatttccttgagatctggttttcgattttaatttggatggaatttagatcactaaatcttttggttttactagaatcgatctaagttgaccgatcagttagctcgattatagctaccagaatttggcttgaccttgatggagatccgatccatgttcatgttacctgggaaatgatttgtatatgtgaattgaaatctgactactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggttttcggttttctatttgactgtattgaactctttttgtgtgtttgcagctcataaacc

SEQ ID NO: 4 - DNA, 910 bp, Artificial Sequence
  Description of Artificial SequenceActin based promoter
tcgagcaggtcacagtcatgaagccatcaaagcaaaagaactaatccaagggctgagatgattaattagtttaaaaattagataacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagagagagaaagtaagagataatccaggttctccgttttgaatcttcctcaatctcatcttcttccgctctttctttccaaggtaataggaactttctggatctactttatttgctggatctcgatcttgttttctcaatttccttgagatctggttttcgtttaatttggatggaatttagatcactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaaccatggctgaggctgatgatattcaaccaatcgtgtgtgacaatggtactggtatggtg

SEQ ID NO: 5 - DNA, 1259 bp, Artificial Sequence
  Description of Artificial SequenceActin based promoter
caactatttttatgtatgcaagagtttcatcttgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggaaatgaaaagtttaagataaccactaaaaaaacggagctgtcatgtaacacgcggatcgagcaggtcacagtcatgaagccatcaaagcaaaagaactaatccaagggctgagatgattaattagtttaaaaattagataacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagagagagaaagtaagagataatccaggttctccgttttgaatcttcctcaatctcatcttcttccgctctttctttccaaggtaataggaactttctggatctactttatttgctggatctcgatcttgttttctcaatttccttgagatctggttttcgtttaatttggatggaatttagatcactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaaccatggctgaggctgatgatattcaaccaatcgtgtgtgacaatggtactggtatggtg

SEQ ID NO: 6 - DNA, 1342 bp, Artificial Sequence
  Description of Artificial SequenceActin based promoter
ccatttttcttttgcatcaactatttttatgtatgcaagagtttcatcttgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaatttgtagaaatgatacaattctaatagaaaacgaattaaattacgttgaattgtatgaaatctaattgaacaagccaaccacgacgacgactaacgttgcctggaaatgaaaagtttaagataaccactaaaaaaacggagctgtcatgtaacacgcggatcgagcaggtcacagtcaatccatgaagccatcaaagcaaaagaactaatccaagggctgagatgattaattagtttaaaaattagataacacgagggaaaaggctgtctgacagccaggtcacgttatactctttacctgtggtcgaaatgattcgtgtctgtcgattttaattatttccaaaattgattttgaaaggccgaaaataaagttgtaagagataaacccgcctatataaattcatcaacactcgatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttcgtggatctactttatttgctggatctcgatcttgttttctcaatttccttgagatctggttttcgattttaatttggatggaatttagatcactaaatcttttggttttactagaatcgatctaagttgaccgatcagttagctcgattatagctaccagaatttggcttgaccttgatggagatccgatccatgttcatgttacctgggaaatgatttgtatatgtgaattgaaatctgactactgttgaagttagattattgactgtaactgtcaatgttagattattgactgtaactgtttaagttagatgaagtttgtgtatagattcttcgaaactttaggatttgtagtgtcgtacgttgaacagaaagctatttctgattcaatcagggttttcggttttctatttgactgtattgaactctttttgtgtgtttgcagctcataaaccatggctgaggctgatgatattcaaccaatcgtgtgtgacaatggtactggtatggtg

SEQ ID NO: 7 - DNA, 74 bp, Artificial Sequence
  Description of Artificial Sequence DNA fragments for assembling an actin-based promoter
ttaattaatcgagcaggtcacagtcatgaagccatcaaagcaaaagaactaatccaagggctgagatgattaat

SEQ ID NO: 8 - DNA, 77 bp, Artificial Sequence
  Description of Artificial Sequence DNA fragments for assembling an actin-based promoter
tagtttaaaaattagataacacgagggaaaaggctgtctgacagccaggtcacgttatctttacctgtggtcgaaat

SEQ ID NO: 9 - DNA, 79 bp, Artificial Sequence
  Description of Artificial Sequence DNA fragments for assembling an actin-based promoter
gattcgtgtctgtcgattttaattatttttttgaaaggccgaaaataaagttgtaagagataaacccgcctatataaat

SEQ ID NO: 10 - DNA, 67 bp, Artificial Sequence
  Description of Artificial Sequence DNA fragments for assembling an actin-based promoter
tcatatattttcctctccgctttgaattgtctcgttgtcctcctcactttcatcagccgttttgaat

SEQ ID NO: 11 - DNA, 73 bp, Artificial Sequence
  Description of Artificial Sequence DNA fragments for assembling an actin-based promoter
ctccggcgacttgacagagaagaacaaggaagagagagaaagtaagagataatccaggttctccgttttgaat

SEQ ID NO: 12 - DNA, 97 bp, Artificial Sequence
  Description of Artificial Sequence DNA fragments for assembling an actin-based promoter
cttcctcaatctcatcttcttccgctctttctttccaaggtaataggaactttctggatctactttatttgctggatctcgatcttgttttctcaat

SEQ ID NO: 13 - DNA, 71 bp, Artificial Sequence
  Description of Artificial Sequence DNA fragments for assembling an actin-based promoter
ttccttgagatctggttttcgtttaatttggatggaatttagatcactaaatcttttggttttactagaat

SEQ ID NO: 14 - DNA, 94 bp, Artificial Sequence
  Description of Artificial Sequence DNA fragments for assembling an actin-based promoter
cgatctaagttgaccgatcagttagctcgattatagctaccagaatttggcttgaccttgatggagagatccatgttcatgttacctgggaaat

SEQ ID NO: 15 - DNA, 63 bp, Artificial Sequence
  Description of Artificial Sequence DNA fragments for assembling an actin-based promoter
gatttgtatatgtgaattgaaatctgaactgttgaagttagattattgactgtaactgtcaat

SEQ ID NO: 16 - DNA, 41 bp, Artificial Sequence
  Description of Artificial Sequence DNA fragments for assembling an actin-based promoter
gttagattattgactgtaactgtttaagttagatgaagttt

SEQ ID NO: 17 - DNA, 79 bp, Artificial Sequence
  Description of Artificial Sequence DNA fragments for assembling an actin-based promoter
gtgtatagattcttcgaaactttaggatttgtagtgtcgtacgttgaacagaaagctatttctgattcaatcagggttt

SEQ ID NO: 18 - DNA, 54 bp, Artificial Sequence
  Description of Artificial Sequence DNA fragments for assembling an actin-based promoter
atttgactgtattgaactctttttgtgtgtttgcagctcataaaccggcgcgcc